(19) United States
(12) Patent Application Publication
(10) Pub. No.: US 2024/0050531 A1
(43) Pub. Date: Feb. 15, 2024

(54) A METHOD FOR REVERSING AGING BRAIN FUNCTIONAL DECLINE

(71) Applicant: THE CHINESE UNIVERSITY OF HONG KONG, Hong Kong (CN)

(72) Inventors: Ho KO, Hong Kong (CN); Zhongqi LI, Taiyuan (CN); Xinyi CHEN, Hong Kong (CN); Si Long VONG, Hong Kong (CN); Lei ZHAO, Hong Kong (CN); Chung Tong Vincent MOK, Hong Kong (CN); Junzhe HUANG, Hong Kong (CN); Yik Chun Leo YAN, Hong Kong (CN); Hei Ming LAI, Hong Kong (CN)

(21) Appl. No.: 18/257,745

(22) PCT Filed: Dec. 16, 2021
(86) PCT No.: PCT/CN2021/138827

§ 371 Date: Jun. 15, 2023

Related U.S. Application Data
(60) Provisional application No. 63/126,122, filed on Dec. 16, 2020.

(51) Int. Cl. A61K 38/26; A61P 25/28; A61K 31/4439; A61K 31/437; A61K 31/444

(52) U.S. Cl. CPC A61K 38/26; A61P 25/28; A61K 31/4439; A61K 31/437; A61K 31/444

(57) ABSTRACT

Provided herein are methods to treat brain functional decline during aging, which involve administration of glucagon-like peptide-1 receptor (GLP-1R) agonists (GLP-1RAs) that treat the aging-associated changes of the brain. The GLP-1R agonist is exenatide, liraglutide, lixisenatide, albiglutide, dulaglutide, semaglutide, taspoglutide, PF-06882961, OWL-833, TTP-273, or any other molecule that activates GLP-1R.

### CROSS-REFERENCE TO RELATED APPLICATION

This application claims the benefit of U.S. Ser. No. 63/126,122, filed Dec. 16, 2020, which is hereby incorporated by reference in its entirety including any tables, figures, or drawings.

### BACKGROUND OF THE INVENTION

Aging has been considered an irreversible process. Nearly all cellular processes have been implicated or impacted by aging, ranging from metabolism, stress response, immune responses, cellular senescence, to gene expression and genomic stability (Almanzar et al., A single-cell transcriptomic atlas characterizes ageing tissues in the mouse. Nature 583, 590-595 (2020)). These complex molecular alterations presumably lead to an alteration of cellular states and compositions in many body organs, manifesting as age-related functional decline. In the brain, hallmarks of aging include (1) mitochondrial dysfunction; (2) dysregulated energy metabolism; (3) intracellular accumulation of oxidatively damaged proteins, nucleic acids, and lipids; (4) impaired cellular “waste disposal” mechanisms (i.e. autophagy, lysosome and proteasome functionality); (5) impaired adaptive stress response signaling; (6) compromised DNA repair; (7) aberrant neuronal network activity; (8) dysregulated neuronal calcium signaling and handling; (9) stem cell exhaustion; and (10) inflammation (M P Mattson and T V Arumugan, Hallmarks of Brain Aging: Adaptive and Pathological Modification by Metabolic States. Cell Metabolism 27: 1176-1199 (2018)). Given the complexity of biological changes involved and a lack of easily targetable sets of driving pathways, anti-aging pharmacotherapy is considered highly challenging. The molecular and cellular alterations in the aging brain lead to the decline of its functions and render it vulnerable to stroke and neurodegeneration (e.g. Alzheimer's and Parkinson's diseases). As human lifespan increases, the population suffering from aging-associated brain conditions increase dramatically. Slowing down or even reversing these alterations in the aging brain may provide a strategy for primary prevention and even the treatment of these conditions.

Glucagon-like peptide-1 (GLP-1) is a peptide hormone produced peripherally by the intestinal L-cells for potentiating glucose-dependent insulin release, and centrally in the brain by preproglucagon neurons in the nucleus tractus solitarii (Holt et al., Preproglucagon Neurons in the Nucleus of the Solitary Tract Are the Main Source of Brain GLP-1, Mediate Stress-Induced Hypophagia, and Limit Unusually Large Intakes of Food. Diabetes 68, 21-33 (2018)). In the past decade, multiple pharmacokinetically optimized GLP-1 receptor (GLP-1R) agonists (GLP-IRAs) have been approved for the clinical treatment of diabetes mellitus. Apart from treatment of diabetes mellitus, evidence in cellular and animal models of neurodegeneration supports neurotrophic and neuroprotective roles of GLP-1R stimulation, as well as in increasing neurogenesis (Salcedo et al., Neuroprotective and neurotrophic actions of glucagon-like peptide-1: An emerging opportunity to treat neurodegenerative and cerebrovascular disorders. British Journal of Pharmacology. Vol. 166 1586-1599 (2012); Grieco et al. Glucagon-Like Peptide-1: A Focus on Neurodegenerative Diseases. Frontiers in Neuroscience vol. 13 (2019); During et al. Glucagon-like peptide-1 receptor is involved in learning and neuroprotection. Nature Medicine 9, 1173-1179 (2003); U.S. Pat. No. 6,969,702B2—Compounds and methods for increasing neurogenesis; Isacson et al., The glucagon-like peptide 1 receptor agonist exendin-4 improves reference memory performance and decreases immobility in the forced swim test. European Journal of Pharmacology 650, 249-255 (2011))(1,2,3-5); During et al., Glucagon-like peptide-1 receptor is involved in learning and neuroprotection. Nature Medicine 9, 1173-1179 (2003)) have shown that GLP-1R is involved in learning and neuroprotection. Intracerebroventricular (i.c.v) administration of GLP-1 and [Ser(2)]exendin(1-9) to 8 weeks old rats enhance associative and spatial learning through GLP-1R. Peripheral administration of [Ser(2)]exendin(1-9) is also active. Isacson et al. (Isacson et al., The glucagon-like peptide 1 receptor agonist exendin-4 improves reference memory performance and decreases immobility in the forced swim test. European Journal of Pharmacology 650, 249-255 (2011)) have shown that intraperitoneal (i.p.) injection of exendin-4 improves hippocampus-associated reference memory performance and decreases immobility in the forced swim test. Remarkably, recent clinical studies provided compelling evidence that GLP-1RAs exhibit neuroprotective effects beyond that conferred by glycemic control, reducing the incidences of cognitive decline and Parkinson's disease (PD) in diabetic patients (Cukierman-Yaffe et al., Effect of dulaglutide on cognitive impairment in type 2 diabetes: an exploratory analysis of the REWIND trial. Lancet Neurology 19, 582-590 (2020); Brauer et al., Diabetes medications and risk of Parkinson's disease: a cohort study of patients with diabetes. Brain (2020) https:/doi.org/10.1093/brain/awaa262). Additionally, GLP-1RAs may slow the progression of established Alzheimer's disease (AD) and PD in non-diabetic patients (Gejl, et al., In Alzheimer's Disease, 6-Month Treatment with GLP-1 Analog Prevents Decline of Brain Glucose Metabolism: Randomized, Placebo-Controlled, Double-Blind Clinical Trial. Frontiers in Aging Neuroscience 8, 108 (2016); Athauda et al., Exenatide once weekly versus placebo in Parkinson's disease: a randomised, double-blind, placebo-controlled trial. Lancet (London, England) 390, 1664-1675 (2017)). Mechanistically, apart from alleviating neuroinflammation in animal models of neurodegeneration (Cai et al., Lixisenatide reduces amyloid plaques, neurofibrillary tangles and neuroinflammation in an APP/PS1/tau mouse model of Alzheimer's disease. Biochem Bioph Res Co 495, 1034-1040 (2018); Yun et al., Block of A1 astrocyte conversion by microglia is neuroprotective in models of Parkinson's disease. Nature Medicine 24, 931-938 (2018)); Zhao et al. demonstrated that treatment with exenatide (a GLP-1RA) partially reverses age-related transcriptomic changes in brain endothelial cells (ECs) and reduces nonspecific blood-brain barrier (BBB) leakage (Zhao et al., Pharmacologically reversible zonation-dependent endothelial cell transcriptomic changes with neurodegenerative disease associations in the aged brain. Nat Commun 11, 4413 (2020)).

Aging is the process of becoming older. This term refers especially to mammals. In the broader sense, aging can refer to a single cell or to an organ within an organism. In humans, aging represents the accumulation of changes in a human being over time. Aging is the main risk factor for the prevalent diseases of developed countries: cancer, cardiovascular disease and neurodegenerative diseases (Niccoli & Partridge. Ageing as a risk factor for disease. Current Biology vol. 22: R741—R752 (2012)). The causes of aging remain unclear. One of the current theories is the damage theory, whereby accumulated damages result in a progressive loss of physiological integrity, leading to impaired function and increased vulnerability to death (Lopez-Otin et al. The hallmarks of aging. Cell vol. 153: 1194 (2013)).

Aging involves nearly all cell types and cellular processes. Furthermore, the signatures of aging brain imposed thereof are different from those in neurodegenerative diseases that have particular manifestations, for example, AD, PD, and Huntington's disease. Given the complexity of biological changes involved and a lack of easily targetable sets of driving pathways, anti-aging pharmacotherapy is considered highly challenging, if feasible at all. Thus, there is a need for slowing down or even reversing transcriptomic and functional alterations in the aging brain.

### BRIEF SUMMARY OF THE INVENTION

Described herein are methods for treating a subject with aging-associated brain conditions. Aspects of the methods include administering a GLP-1R agonist to a subject in need thereof, such as, for example, an individual at risk of developing or suffering from aging-associated brain conditions. Aging-associated brain conditions include, for example, aging-associated cognitive impairment and structural, functional or molecular changes of the brain.

In certain embodiments, the methods involve treatment of a subject that can be at risk of developing or suffering from brain conditions that are caused by natural aging.

In certain embodiments, the subject can be at risk of developing or suffering from aging-associated brain conditions has transcriptomic and functional changes across multiple cell types within the brain, including neurons, such as, for example, mature neuron (mNeur) and immature neuron (imNeur); glial cells, such as, for example, astrocyte (AC), oligodendrocyte precursor cell (OPC), microglia (MG), and oligodendrocyte (OLG); mural cells, such as, for example, pericytes (PC) and smooth muscle cell (SMC) cells; choroid plexus cells (CPC); hemoglobin-expressing vascular cells (Hb_EC); and monocytes (MNC).

In certain embodiments, the subject can be at risk of developing or suffering from aging-associated brain conditions that can be treated with a GLP-1R agonist (GLP-1RA), such as, for example, exenatide, liraglutide, lixisenatide, albiglutide, dulaglutide, semaglutide, taspoglutide, PF-06882961, OWL-833, and/or TTP-273.

In certain embodiments, the GLP-1RA treatment can reverse and/or inhibit the transcriptomic and functional changes presented in multiple cell types within brain, including neurons, such as, for example, mature neuron (mNeur) and immature neuron (imNeur); glial cells, such as, for example, astrocyte (AC), oligodendrocyte precursor cell (OPC), microglia (MG), and oligodendrocyte (OLG)); mural cells, such as, for example, pericytes (PC) and smooth muscle cell (SMC) cells; choroid plexus cells (CPC); hemoglobin-expressing vascular cells (Hb_EC); and monocytes (MNC).

### BRIEF DESCRIPTION OF THE DRAWINGS

FIG. 1A shows UMAP visualization of the major cell type clusters identified and analyzed in mouse brains. AC: astrocyte; OPC: oligodendrocyte precursor cell; MG: microglia; MAC: perivascular macrophage; OLG: oligodendrocyte; SMC: smooth muscle cell; PC: pericyte; EC: endothelial cell. Numbers in brackets: cell numbers for the respective cell types.

FIG. 1B shows UMAP visualization of the single-cell transcriptomes from young adult mouse brains. Numbers in brackets: cell numbers for the group (n=3 animals for the group).

FIG. 1C shows UMAP visualization of the single-cell transcriptomes from aged adult mouse brains. Numbers in brackets: cell numbers for the group (n=3 animals for the group).

FIG. 1D shows UMAP visualization of the single-cell transcriptomes from GLP-1RA (exenatide)-treated aged mouse brains. Numbers in brackets: cell numbers for the group (n=3 animals for the group).

FIG. 2A shows Age-related expression changes (x-axis) plotted against post-GLP-1RA treatment expression changes (y-axis) in glial (AC: astrocyte; OPC: oligodendrocyte precursor cell; MG: microglia; and OLG: oligodendrocyte), vascular (EC: endothelial cell; PC: pericyte; SMC: smooth muscle cell) cell types, and MAC (perivascular macrophage). Each dot represents one differentially expressed gene (DEG). Grey lines: lines of best fit by linear regression.

FIG. 2B shows proportions of DEGs reversed and the slopes of lines of best fit by linear regression shown in FIG. 2A in the different cell types.

FIG. 3 shows age-related expression changes (x-axis) plotted against post-GLP-1RA treatment expression changes (y-axis) in neurons (mNeur: mature neuron; imNeur: immature neuron; MG: microglia; and OLG: oligodendrocyte), monocyte (MNC), choroid plexus cell (CPC), and hemoglobin-expressing vascular cell. Each dot represents one differentially expressed gene (DEG). Grey lines: lines of best fit by linear regression.

FIG. 4 shows functional pathways with significant enrichment among the most prominent age-related expression changes reversed by GLP-1RA treatment in the different brain cell types.

FIG. 5 shows expression changes of selected functionally important genes in AC, MG and SMC, in aging and after GLP-1RA treatment.

FIG. 6 shows improvement of spatial memory of aged mice treated with GLP-1RA in Y maze test. 15 to 17 months old C57BL/6 mice were treated daily with saline vehicle or 5 nmol/kg bw of exenatide for 2-4 months.

### DETAILED DISCLOSURE OF THE INVENTION

### Definitions

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19 and 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein a “reduction” means a negative alteration, and an “increase” means a positive alteration, wherein the negative or positive alteration is at least 0.001%, 0.01%, 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, the term “pharmaceutically acceptable” means compatible with the other ingredients of a pharmaceutical composition and not deleterious to the recipient thereof.

As used herein, the terms “therapeutically-effective amount,” “therapeutically-effective dose,” “effective amount,” and “effective dose” are used to refer to an amount or dose of a compound or composition that, when administered to a subject, is capable of treating or improving a condition, disease, or disorder in a subject or that is capable of providing enhancement in health or function to an organ, tissue, or body system. In other words, when administered to a subject, the amount is “therapeutically effective.” The actual amount will vary depending on a number of factors including, but not limited to, the particular condition, disease, or disorder being treated or improved; the severity of the condition; the particular organ, tissue, or body system of which enhancement in health or function is desired; the weight, height, age, and health of the patient; and the route of administration.

As used herein, “treatment”, “treating”, “palliating” and “ameliorating” (and grammatical variants of these terms), as used herein, are used interchangeably. These terms refer to an approach for obtaining beneficial or desired results including, but not limited to, therapeutic benefits. A therapeutic benefit is achieved with the eradication or amelioration of one or more of the physiological symptoms associated with the underlying disease such that an improvement is observed in the subject, notwithstanding that the subject may still be afflicted with the underlying disease. A treatment includes delaying the appearance of a disease or condition, delaying the onset of symptoms of a disease or condition, slowing, halting, or reversing the progression of a disease or condition, or any combination thereof. The terms refer to eradicating, reducing, ameliorating, or reversing a sign or symptom of a health condition, disease or disorder to any extent, and include, but do not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

As used herein, the term “elderly” refers to a subject in an age group that is past middle age. Elderly refers to a specific age group, such as, for example, subjects 50 years old or older, 60 years old or older, 65 years old or older, 70 years old or older, or 75 years old or older. The elderly subject is preferably a mammal, more preferably a human, and even more preferably a human adult, as well as at least 50, 55, 60, 65, or 70 years old. More preferably, the patient is an elderly (adult) human patient who is aged or older. The elderly patient may be male or female.

As used herein, the phrase “executive functions” refers to a set of cognitive abilities that control and regulate other abilities and behaviors. Executive functions are high-level abilities that influence more basic abilities like attention, memory and motor skills. The executive functions are necessary for goal-directed behavior, and include the ability to initiate and stop actions, to monitor and change behavior as needed, and to plan future behavior when faced with novel tasks and situations. Executive functions allow subjects to anticipate outcomes and adapt to changing situations. The ability to form concepts and the ability to think abstractly are often considered components of executive function.

### Aging-Associated Brain Conditions

Together with other organs, the functions of the brain decline progressively during aging. Brains in the aged population are at risk of developing or suffering from aging-associated conditions, which manifest as decrements in learning and memory, attention, decision-making speed, sensory perception, and motor coordination.

In certain embodiments, a subject that will benefit from treatment as disclosed here include individuals that are about at least 50 years old, 60 years old, 70 years old, 80 years old, 90 years old, and usually no older than 100 years old; between the ages of about 50 and 100; or about 50, about 55, about 60, about 65, about 70, about 75, about 80, about 85, about 90, about 95 or about 100 years old, and are at risk of developing or suffering from aging-associated conditions, such as, for example, cognitive impairment and/or measurable structural, functional or molecular changes of the brain.

In certain embodiments, cognitive impairment can comprise various brain conditions, such as, for example, attention and concentration; learning tasks and concepts; memory; information processing; visuospatial function; producing speech; understanding language; verbal fluency; problem solving; decision making; and executive functions. Cognitive impairment can be assessed by numbers of tests, such as, for example, memory, recall, visuospatial awareness, verbal fluency, expressive language, executive function, gait, and dual-task. Examples of tests used to detect such differences include the mini-mental state examination (MMSE), the Montreal Cognitive Assessment (MoCA) and its variants, the Alzheimer's Disease Assessment Scale-Cognitive Subscale (ADAS-Cog) and the Clinical Dementia Rating (CDR) scale. A score satisfying any of the criteria: 26 or below on MMSE, or below on MoCA, 12 or more on ADAS-Cog, 0.5 or more on CDR are considered cognitively impaired.

In certain embodiments, structural changes of the brain can be changes in brain volume, i.e., 5%, 10%, 15%, 20% or more decrease, structure of grey matter, structure of white matter, structure of the ventricular system, structure and integrity of neurovascular system. Structural changes in the brain can be measured by an imaging method such as, for example, magnetic resonance imaging (MRI), computed tomography (CT), or ultrasonography (US). Age-related decline in these measures are defined as alterations in numerical values and/or patterns obtained by the respective imaging or recording methods, that reflect changes in structure of volume, which fall at the extrema of distributions (e.g. top or bottom 5%, 10%, 20%, 25%, 30%) for whole population or population of the same age, or exhibit changes on repeated measurement from the same subject (e.g. 5%, 10%, 15%, 20%, 25% or more) over time.

In certain embodiments, functional changes of the brain can refer to measurable changes in the brain reflecting altered states of neuronal activity, metabolism, or neurovascular functions. Functional changes of the brain can be measured by an imaging method such as, for example, functional magnetic resonance imaging (fMRI), magnetic resonance imaging (MRSI); hyperpolarized carbon-13 (¹³C) magnetic resonance spectroscopic imaging (MRSI), ultrasonography (US), positron emission tomography (PET), or single-photon emission computerized tomography (SPECT). Functional changes of the brain can also be recorded by an electrophysiological method such as, for example, electroencephalography (EEG); intracortical electrode recording; and deep-brain electrode recording or a magnetic field-based recording method, including magnetoencephalography (MEG). Age-related decline in these measures are defined as alterations in numerical values and/or patterns obtained by the respective imaging or recording methods, that reflect neuronal activity, metabolism or neurovascular functions, which fall at the extrema of distributions (e.g. top or bottom 5%, 10%, 20%, 25%, 30%) for whole population or population of the same age, or exhibit changes on repeated measurement from the same subject (e.g. 5%, 10%, 15%, 20%, 25% or more) over time.

In certain embodiments, molecular changes of the brain can refer to alterations at the molecular level measurable in brain tissues or blood fluids, such as, for example, changes in gene expression; transcription of DNA; translation of RNA; locations of DNA, RNA or protein in brain cells or tissue; and/or secretion or release of proteins, DNA, RNA, mitochondria, cellular components, and/or mitochondrial components into blood fluids. The body fluid can be cerebrospinal fluid (CSF), blood, or plasma. Age-related changes in these measures are defined as alterations in numerical values and/or patterns obtained by the respective methods, that reflect molecular changes in the brain, which fall at the extrema of distributions (e.g. top or bottom 5%, 10%, 20%, 25%, 30%) for whole population or population of the same age, or exhibit changes on repeated measurement from the same subject (e.g. 5%, 10%, 15%, 20%, 25% or more) over time.

### GLP-1R Agonist

In certain embodiments, GLP-1R agonists can be used in methods of the subject invention. GLP-1R agonists are agonists of the GLP-1 receptor. GLP-1, a natural agonist of GLP-1R, has a short duration of action. Several pharmacologically optimized GLP-1R agonists have been approved or under development for the clinical treatment of diabetes mellitus, or neurodegenerative diseases, for example, Alzheimer's disease and Parkinson's disease.

The activity of a GLP-1R agonist can be determined by assays of well-studied downstream signaling and regulatory pathways linked to GLP-1R activation, such as, for example, increased cAMP production, induced phosphorylation of ERK1/2, enhanced intracellular mobilization of calcium, and recruitment of beta-arrestin-1 and beta-arrestin-2.

Some non-limiting examples of GLP-1R agonists include exenatide, liraglutide, lixisenatide, albiglutide, dulaglutide, semaglutide, taspoglutide, PF-06882961, OWL-833, and TTP-273.

### Dosage and Administration

In certain embodiments, the methods described herein provide a method for the treatment of aging-associated brain conditions. In one embodiment, the subject can be a mammal. In another embodiment, the subject can be a human, although the invention is effective with respect to all mammals. The method can comprise administering to the subject an effective amount of a pharmaceutical composition comprising a GLP-1R agonist that reverses the transcriptomic and functional changes in the aging brain The subject composition can further comprise one or more pharmaceutically acceptable carriers and/or excipients, and can be formulated into preparations, for example, solid, semi-solid, liquid, or gaseous forms, such as tablets, capsules, powders, granules, ointments, solutions, suppositories, injections, inhalants, and aerosols. Pharmaceutically acceptable carriers are well known in the art and include, for example, aqueous solutions, such as water or physiologically buffered saline or other solvents or vehicles, such as polyethylene glycol, Tween-20 or olive oil, or injectable organic esters. The dosage range for the agonist can depend on the potency. In certain embodiments, large amounts of an agonist can produce a desired effect, such as, for example, a reversal in the transcriptomic and functional changes in any major cell type in the aging brain or a reversal in structural and/or functional changes of an aging brain. The dosage should not be so large as to cause adverse side effects. Generally, the dosage will vary with the agents to be used. Additionally, the age, condition, and sex of the subject can be determined by one of the skill in the art and used to determine dosage. The dosage can also be adjusted by the individual physician in the event of any complications.

In one embodiment, the composition is formulated as an orally-consumable product, such as a food item, capsule, pill, or drinkable liquid. An orally deliverable health-promoting compound is any physiologically active substance delivered via initial absorption in the gastrointestinal tract or into the mucus membranes of the mouth. The composition can also be formulated as a solution that can be administered via, for example, injection, which includes intravenously, intraperitoneally, intramuscularly, intrathecally, or subcutaneously. In other embodiments, the subject composition is formulated to be administered via the skin through a patch or directly onto the skin for local or systemic effects. The compositions can also be administered sublingually, buccally, rectally, or vaginally. Furthermore, the compositions can be sprayed into the nose for absorption through the nasal membrane, nebulized, inhaled via the mouth or nose, or administered in the eye or ear.

Orally-consumable products, according to the invention, are any preparations or compositions suitable for consumption, for nutrition, for oral hygiene, or for pleasure and are products intended to be introduced into the human or animal oral cavity, to remain there for a certain period of time, and then either to be swallowed (e.g., food ready for consumption or pills) or to be removed from the oral cavity again (e.g., chewing gums or products of oral hygiene or medical mouth washes). While an orally-deliverable pharmaceutical can be formulated into an orally consumable product, and an orally consumable product can comprise an orally deliverable pharmaceutical, the two terms are not meant to be used interchangeably herein.

Orally consumable products include all substances or products intended to be ingested by humans or animals in a processed, semi-processed, or unprocessed state. This also includes substances that are added to orally consumable products (particularly food and pharmaceutical products) during their production, treatment, or processing and intended to be introduced into the human or animal oral cavity.

Orally consumable products can also include substances intended to be swallowed by humans or animals and then digested in an unmodified, prepared, or processed state. The orally consumable products, according to the invention, also include casings, coatings, or other encapsulations that are intended to be swallowed together with the product or for which swallowing is to be anticipated.

In one embodiment, the orally consumable product is a capsule, pill, syrup, emulsion, or liquid suspension containing a desired orally deliverable substance. In one embodiment, the orally consumable product can comprise an orally deliverable substance in powder form, which can be mixed with water or another liquid to produce a drinkable orally consumable product.

Carriers and/or excipients, according the subject invention, can include any and all solvents, diluents, buffers (such as neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g. carbomer, gelatin, or sodium alginate), coatings, preservatives (e.g., Thimerosal, benzyl alcohol, polyquaterium), antioxidants (e.g., ascorbic acid, sodium metabisulfite), tonicity controlling agents, absorption delaying agents, adjuvants, bulking agents (e.g., lactose, mannitol), and the like. The use of carriers and/or excipients in the field of drugs and supplements is well known. Except for any conventional media or agent that is incompatible with the target health-promoting substance or with the adjuvant composition, carrier or excipient use in the subject compositions may be contemplated.

In one embodiment, the composition can be made into aerosol formulations so that, for example, it can be nebulized or inhaled. Suitable pharmaceutical formulations for administration in the form of aerosols or sprays are, for example, solutions, suspensions, or emulsions. Formulations for oral or nasal aerosol or inhalation administration may also be formulated with illustrative carriers, including, for example, saline, polyethylene glycol or glycols, DPPC, methylcellulose, or in mixture with powdered dispersing agents or fluorocarbons. Aerosol formulations can be placed into pressurized propellants, such as dichlorodifluoromethane, propane, nitrogen, fluorocarbons, and/or other solubilizing or dispersing agents known in the art. Illustratively, delivery may be by use of a single-use delivery device, a mist nebulizer, a breath-activated powder inhaler, an aerosol metered-dose inhaler (MDI), or any other of the numerous nebulizer delivery devices available in the art. Additionally, mist tents or direct administration through endotracheal tubes may also be used.

In one embodiment, the composition can be formulated for administration via injection, for example, as a solution or suspension. The solution or suspension can comprise suitable non-toxic, parenterally-acceptable diluents or solvents, such as mannitol, 1,3-butanediol, water, Ringer's solution, or isotonic sodium chloride solution, or suitable dispersing or wetting and suspending agents, such as sterile, non-irritant, fixed oils, including synthetic mono- or diglycerides, and fatty acids, including oleic acid. One illustrative example of a carrier for intravenous use includes a mixture of 10% USP ethanol, 40% USP propylene glycol or polyethylene glycol 600, and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance is an acceptable isotonic solution, such as 5% dextrose or 0.9% sodium chloride; or 0.01-0.2% dipalmitoyl diphosphatidylcholine in USP WFI and 1 to 10% squalene or parenteral vegetable oil-in-water emulsions.

In one embodiment, the adjuvant composition can be formulated for administration via topical application onto the skin, for example, as topical solutions, which include rinse, spray, drop, lotion, gel, ointment, cream, foam, powder, solid, sponge, tape, vapor, paste, tincture, or a transdermal patch. Suitable formulations of topical applications can comprise, in addition to any of the pharmaceutically active carriers, emollients, such as carnauba wax, cetyl alcohol, cetyl ester wax, emulsifying wax, hydrous lanolin, lanolin, lanolin alcohols, microcrystalline wax, paraffin, petrolatum, polyethylene glycol, stearic acid, stearyl alcohol, white beeswax, or yellow beeswax. Additionally, the compositions may contain humectants, such as glycerin, propylene glycol, polyethylene glycol, sorbitol solution, and 1,2,6 hexanetriol or permeation enhancers, such as ethanol, isopropyl alcohol, or oleic acid.

Further components can be added to the compositions as are determined by the skilled artisan, for example, buffers, carriers, viscosity modifiers, preservatives, flavorings, dyes, and other ingredients specific for an intended use. One skilled in this art will recognize that the above description is illustrative rather than exhaustive. Indeed, many additional formulations techniques and pharmaceutically-acceptable excipients and carrier solutions suitable for particular modes of administration are well-known to those skilled in the art.

In certain embodiments, the dose will range from about 0.001 mg/kg body weight to about 1 g/kg body weight. In some embodiments, the dose will range from about 0.001 mg/kg body weight to about 0.2 g/kg body weight, from about 0.001 mg/kg body weight to about 0.1 g/kg body weight, from about 0.001 mg/kg body weight to about 20 mg/kg body weight, from about 0.001 mg/kg body weight to about 10 mg/kg body weight, from about 0.001 mg/kg body weight to about 5 mg/kg body weight, from about 0.001 mg/kg body weight to about 2 mg/kg body weight, from about 0.001 mg/kg body weight to about 1 mg/kg body weight, from about 0.001 mg/kg body weight to about 0.2 mg/kg body weight, from about 0.001 mg/kg body weight to about 0.02 mg/kg body weight, from about 0.001 mg/kg body weight to about 0.01 mg/kg body weight. Alternatively, in some embodiments the dose range is from about 0.01 g/kg body weight to about 1 g/kg body weight, from about 0.05 g/kg body weight to about 1 g/kg body weight, from about 0.1 g/kg body weight to about 1 g/kg body weight, from about 0.2 g/kg body weight to about 1 g/kg body weight, from about 0.25 g/kg body weight to about 1 g/kg body weight, from about 0.5 g/kg body weight to about 1 g/kg body weight. In one embodiment, the dose range is from about 5 μg/kg body weight to about 50 μg/kg body weight.

A GLP-1RA that reverses the aging-associated brain conditions can be given multiple times a day, such as, for example, 2-times, 3-times, 4-times, 5-times, 6-times, 7-times, 8-times, 9-times, 10-times, 11-times, or 12-times per day; once a day; less than once a day; once weekly; bi-weekly; monthly; bi-monthly; quarterly; bi-yearly; yearly; or continuously in order to achieve a therapeutically effective dose. Administration of the doses used herein can be repeated for a limited period of time, such as, for example, the doses used herein can be administered daily for several weeks, months or years. Treatment duration depends on the subject's clinical progress and responsiveness to therapy. A therapeutically effective amount is an amount of a GLP-1RA that is sufficient to produce a measurable change of the brain (see “Efficacy Measurement” below). Such effective amounts can be administered in clinical trials as well as animal studies.

GLP-1RAs useful in the invention can be administered orally, intravenously, intranasally, by inhalation, intraperitoneally, intramuscularly, subcutaneously, or intracavity. In one embodiment the GLP-1RAs used herein are administered orally, or subcutaneously to a patient.

### Measurement of Efficacy

In one embodiment, the efficacy of a given treatment can be determined by the improvements in cognitive capabilities, as measured by numbers of tests, such as, for example, memory, recall, visuospatial awareness, verbal fluency, expressive language, executive function, gait, and dual-task, multi-task. These could be reflected as improvements in the values obtained by a cognitive test, including the mini-mental state examination (MMSE), the Montreal Cognitive Assessment (MoCA) and its variants, the Alzheimer's Disease Assessment Scale-Cognitive Subscale (ADAS-Cog) and the Clinical Dementia Rating (CDR) scale. A clinical improvement is defined as a change in numerical values in any of the following forms: an increase in MMSE score, an increase in MoCA score, a decrease in ADAS-Cog score, or a decreased in CDR score. Improvements in cognitive capabilities can also be reflected as the lack of decline or slower decline compared age-match population in any of these scores, such as no changes in the values of MMSE, MoCA, ADAS-Cog or CDR, as with ageing they are expected to continuously decline.

In another embodiment, the efficacy of a given treatment can be determined by the reversal of aging-associated brain structural changes, wherein the changes can be measured by an imaging method such as, for example, magnetic resonance imaging (MRI); computed tomography (CT); and ultrasonography (US). In certain embodiments, the structural changes can be hippocampal atrophy, cortical atrophy, subcortical structural atrophy, cerebellar atrophy, global brain atrophy; microbleeds in the grey matter of the frontal, temporal, parietal or occipital lobes; microbleeds in the white matter of the frontal, temporal, parietal or occipital lobes; lacunes, infarcts, perivascular space, white matter intensity changes in the frontal, temporal, parietal or occipital lobes, and/or aggregation of protein species. Improvements in the structural changes can be in the form of lack of development of new age-related structural changes, static values with time, or altered magnitudes of the measures quantifying the severity of structural changes. The protein species can be amyloid beta, tau, alpha-synuclein, TAR DNA-binding protein 43, and/or prion, occurring in cortical regions, subcortical regions, or in the brainstem.

In another embodiment, the efficacy of a given treatment can be determined by the reversal of aging-associated brain functional changes, wherein the changes can be measured by an imaging method such as, for example, functional magnetic resonance imaging (fMRI); magnetic resonance imaging (MRSI); hyperpolarized carbon-13 (¹³C) magnetic resonance spectroscopic imaging (MRSI); ultrasonography (US); positron emission tomography (PET); and single-photon emission computerized tomography (SPECT). Functional changes of the brain can also be recorded by an electrophysiological method such as, for example, electroencephalography (EEG); intracortical electrode recording; deep-brain electrode recording; or a magnetic field-based recording method, such as, for example, magnetoencephalography (MEG). The functional changes can be altered brain regional activation patterns, neuronal activity, functional connectivity, blood-brain barrier leakage, resting blood flow, glucose consumption, metabolite concentrations, and neurovascular coupling, and/or functional hyperemia. For each of these measures, efficacy is defined as a reversal of the respective values closer to that found in youngers subjects, or the lack of progression or age-related changes in these values that would naturally otherwise occur.

In another embodiment, the efficacy of a given treatment can be determined by the reverse of aging-associated brain molecular changes, wherein the changes can be assessed by measuring molecules from brain tissues or blood fluids, such as, for example, gene expression; transcription of DNA; translation of RNA; locations of DNA, RNA or protein in brain cells or tissue; secretion or release of proteins, DNA, RNA, mitochondria, cellular components, mitochondrial components into blood fluids. The body fluid can be cerebrospinal fluid (CSF), blood and/or plasma.

In certain embodiments, the calculation of differentially expressed genes (DEG) with associated raw P-value, false discovery rate (FDR)-adjusted P-value, and/or magnitude of change expressed in natural log of fold change (1nFC) can be calculated for each cell type. In certain embodiments, a DEG in one in which the P-value is <0.05, preferably a FDR-adjusted P-value <0.05.

In certain embodiments, the genes and subsequent transcribed mRNA and translated protein that can be assessed in the subject methods are immune response-related genes, such as, for example, C1qa, C1qb, C1qc, C4b, B2m, Tap2, H2-D1, H2-K1; synaptic modification-related genes, such as, for example, Sparcl1, Gpc6, Tgfb2, Megf10, Mertk, Chrdl1; homeostatic function-related genes, such as, for example, Kcnj10, Kcnn2, Slc 1 a2, Slc 1 a3, Slc6a1, Slc6a9, Slc6a1l, Slc7a10, Slc7al1, Slc16al, Srebf1, Gja1, Gjb6, Itpr2, Grm3, Gria2, Gabbr1, Gabbr2; homeostatic-related genes in MG cells, such as, for example, Csf2r, and P2ry13; immune activation-related genes in MG cells such as, for example, Appe, Cc13, Cc14, Cd52, Cst7, Fabp5, Tyrobp, Cd14, Cd33, Ifngr1, Ly86, Map4k4; immune response0inhibitaroy genes in MG cells, such as, for example, Cd300a, Il10ra, and Il10rb; calcium signaling-related genes in SMCs, such as, for example, Camk2g, Stim1, Gsn, Δtp2a2, Inpp4b, Mcur1, S100a6, and Tspo; SMC contraction-related genes, such as, for example, Mylk, Itga1, Mgh11, and Sorbs1; and cell adhesion and ECM remodeling in SMCs, such as, Col1a2, Lamb1, Itga7, Jam3, Lamb2, Itgb1, and Bsg.

### Materials and Methods

### Animal Subjects

All experimental procedures were approved in advance by the Animal Research Ethical Committee of the Chinese University of Hong Kong (CUHK) and were carried out in accordance with the Guide for the Care and Use of Laboratory Animals. C57BL/6 J mice were provided by the Laboratory Animal Service Center of CUHK and maintained at a controlled temperature (22-23° C.) with an alternating 12 h light/dark cycle with free access to standard mouse diet and water. The ambient humidity was maintained at <70% relative humidity. Male mice of two age groups (2-3 months old and 18-20 months old) were used for experiments, unless indicated otherwise. For the treatment groups, exenatide (5 nmol/kg bw, Byetta, AstraZeneca LP, Cambridge, UK) or saline vehicle (0.9% w/v sodium chloride) was intraperitoneally (I.P.) administered (volume: 250 μl per 30 g bw) daily for 4-5 weeks prior to experimentation, unless indicated otherwise.

### Brain Tissue Dissociation and Single-Cell Isolation

A dissociation protocol optimized for single brain cell isolation from both young and aged mouse brains (Vanlandewijck, M. et al. Primary isolation of vascular cells from murine brain for single cell sequencing. Protocol Exchange https://doi.org/10.1038/protex.2017.159 (2018)) was adapted for use. Mice were deeply anaesthetized and perfused transcardially with 20 ml of ice-cold phosphate buffered saline (PBS). Mice were then rapidly decapitated, and whole brains (except cerebellum) were immersed in ice-cold Dulbecco's modified Eagle's medium (DMEM, Thermo Fisher Scientific, Waltham, MA). The brain tissues were cut into small pieces and dissociated into single cells using a modified version of the Neural Tissue Dissociation Kit (P) (130-092-628, Miltenyi Biotec, Bergisch Gladbach, Germany). Myelin debris was removed using the Myelin Removal kit II (130-096-733, Miltenyi Biotec) according to the manufacturer's manual. Cell clumps were removed by serial filtration through pre-wetted 70-μm (#352350, Falcon, Corning, NY) and 40-μm (#352340, Falcon) nylon cell strainers. Centrifugation was performed at 300×g for 5 min at 4° C. The final cell pellets were resuspended in 500-1000 μl FACS buffer (DMEM without phenol red (Thermo Fisher Scientific), supplemented with 2% fetal bovine serum (Thermo Fisher Scientific)).

### Single-Cell Library Preparation, Sequencing and Alignment

Single-cell RNA-seq libraries were generated using the Chromium Single Cell 3′ Reagent Kit v3 (10× Genomics, Pleasanton, CA). A single-cell suspension at a density of 500-1000 cells/μL in FACS buffer was added to real-time polymerase chain reaction (RT-PCR) master mix and then loaded together with Single Cell 3′ gel beads and partitioning oil into a Single Cell 3′ Chip, according to the manufacturer's instructions. RNA transcripts from single cells were uniquely barcoded and reverse-transcribed within droplets. cDNA molecules were preamplified and pooled, followed by library construction, according to the manufacturer's instructions. All libraries were quantified by Qubit and RT-PCR on a LightCycler 96 System (Roche Life Science, Penzberg, Germany). The size profiles of the pre-amplified cDNA and sequencing libraries were examined by the Agilent High Sensitivity D5000 and High Sensitivity D1000 ScreenTape Systems (Agilent, Santa Clara, CA), respectively. All single-cell libraries were sequenced with a customized paired-end with single indexing (28/8/91-bp for v3 libraries) format according to the recommendations by 10× Genomics. All single-cell libraries were sequenced on a NextSeq 500 system (Illumina, San Diego, CA) using the NextSeq 500 High Output v2 Kit (Illumina). An average of 5897.25 mean reads per cell (range: 2,340-19,700) were obtained, which detected an average of 2296.01 genes per cell (range: 88-6,062). The library sequencing saturation was on average 68.73%. The data were aligned in Cell Ranger (v3.0.0, 10× Genomics).

### Data Quality Control

Data processing and visualization were performed using the Seurat package (v3.1.5) and custom scripts in R (v3.6.1). The raw count matrix was generated by default parameters (with the mm10 reference genome). There were 106,832 cells in the primary count matrix. Genes expressed by fewer than five cells were removed, leaving 21,259 genes in total.

Among these genes, 3,000 high-variance genes were identified by the Seurat FindVariableFeatures function. The dataset was filtered to exclude low-quality cells by the following criteria: (1)<5% or >95% UMI count or gene count, or (2) proportion of mitochondrial genes >20%. For dimensionality reduction, principal component analysis (PCA) was applied to compute the first 30 top principal components. Clustering was carried out by the Seurat functions FindNeighbors and FindClusters. The FindNeighbors function constructed a shared nearest neighbor (SNN) graph based on the first 50 principal components. Modularity optimization was then performed on the SNN results for clustering (resolution parameter: 1.2). We employed Uniform Manifold Approximation and Projection (UMAP) to visualize the clustering results.

### Cell Type Identification

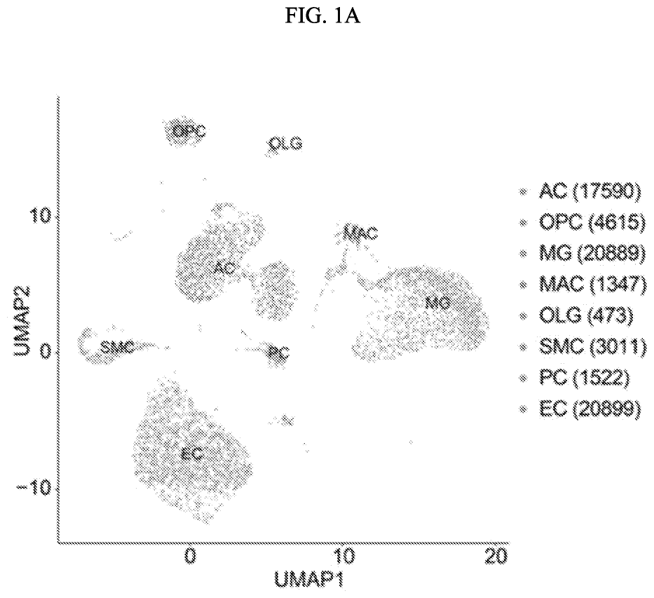

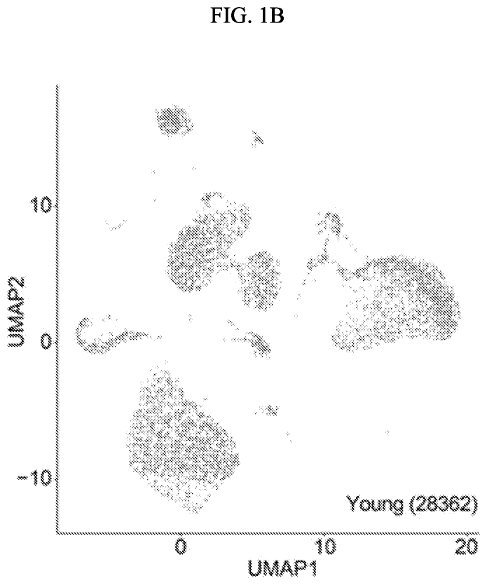

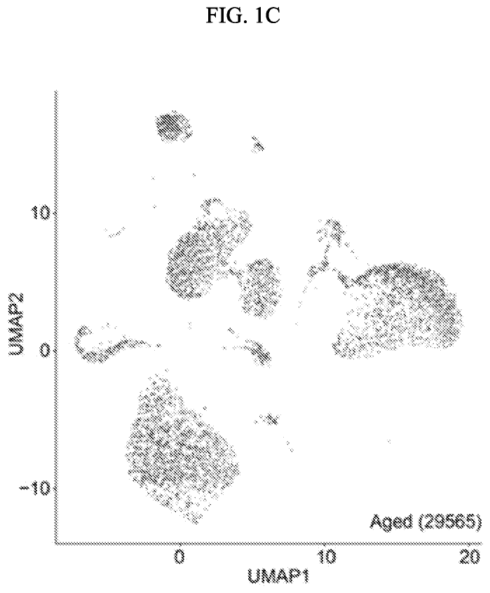

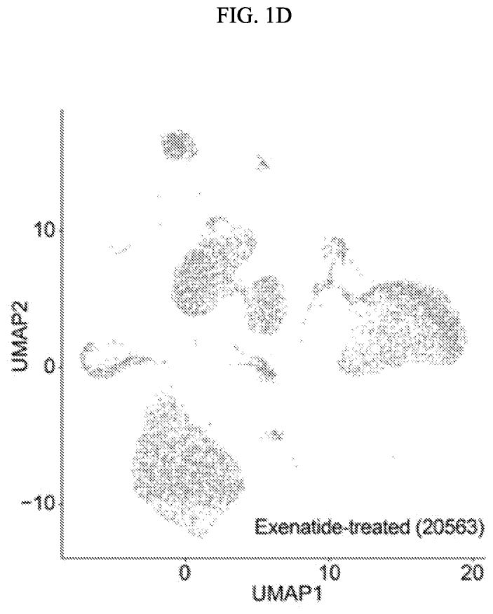

To identify primary cell types, we employed known cell type-specific marker genes and examined their expression levels among all 52 initial clusters included. We further excluded clusters with a dual-high expression of two or more cell type-specific marker genes. These included a cluster with high expression of both endothelial cell and pericyte markers, corresponding to contamination of pericytes by endothelial cell fragments. The remaining clusters were classified into 16 primary cell types (see FIGS. 1A-1D).

### Differentially Expressed Gene Calculation

After quality control and cell type classification, gene count normalization and high variance gene identification were applied to the raw data of 78,490 cells retained for further analysis. The Seurat FindMarkers function and the MAST package (v1.8.2) were employed for calculation of differentially expressed genes (DEG) with associated raw P-value, false discovery rate (FDR)-adjusted P-value and magnitude of change expressed in natural log of fold change (1nFC) for each cell type. We define significant DEGs as those fulfilling FDR-adjusted P-value <0.05.

### Pathway Enrichment and Disease-Association Analysis

We used GeneAnalytics, an online universal gene functional analysis tool, which contained more than a hundred data sources for pathway enrichment analysis. Significant DEGs were converted to human gene orthologs semi-automatically. Pathways with prominent functional implications, including metabolic pathways, immune response and cytokine signaling pathways, respiratory electron transport chain/ATP synthesis and glucose/energy metabolism pathways, gene expression/transcriptional and translational regulatory pathways, calcium and other second messenger signaling pathways, cell type-specific pathways (e.g. SMC contraction), hormonal signaling pathways, cell adhesion and extracellular matrix remodeling-related pathways, were identified manually from literature review, grouped and summarized.

### Y Maze Test

Spatial memory was assessed using the Y maze test. The Y maze comprises three enclosed arms, 30 cm long, 8 cm wide, and 15 cm high made of white acrylic glass, set at an angle of 120° to each other. Visual cues were placed around the maze in the testing room. The test comprises two trials. In the first trial, the mouse was allowed to explore the maze for 5 min with one of the arms closed. The mouse was returned to its home cage away from the test room during the 2 min between trials. In the second trial, the mouse was allowed to explore freely all three arms of the maze for 5 min. The time spent in each arm was registered from video recordings. Arm entry was defined as the main body of the mouse crossed the threshold of the central zone and into the arm. The percentage of time spent in the novel arm (previously closed in the first trial) was calculated.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### EXAMPLES

### Example 1—Exenatide Treatment of Mice

Aging leads to dramatic transcriptomic changes across multiple major brain cell types, including neurons, glial cells, such as, for example, astrocyte (AC), oligodendrocyte precursor cell (OPC), microglia (MG) and oligodendrocyte (OLG)) and mural cells, such as, for example, endothelial cell (EC), pericyte (PC), and smooth muscle cell (SMC). Treatment with exenatide, a GLP-1R agonist, reverses such aging-associated transcriptomic signatures. The age-related expression changes reversed by GLP-1RA encompass shared and cell type-specific functional pathways implicated in aging and neurodegeneration.

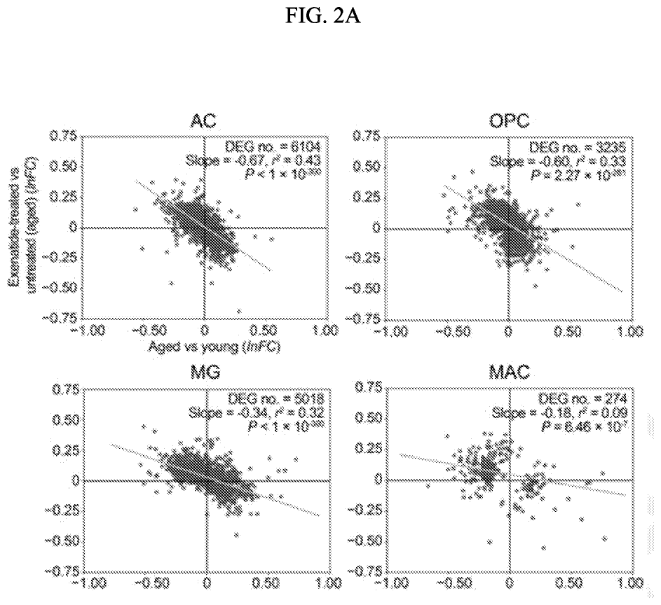

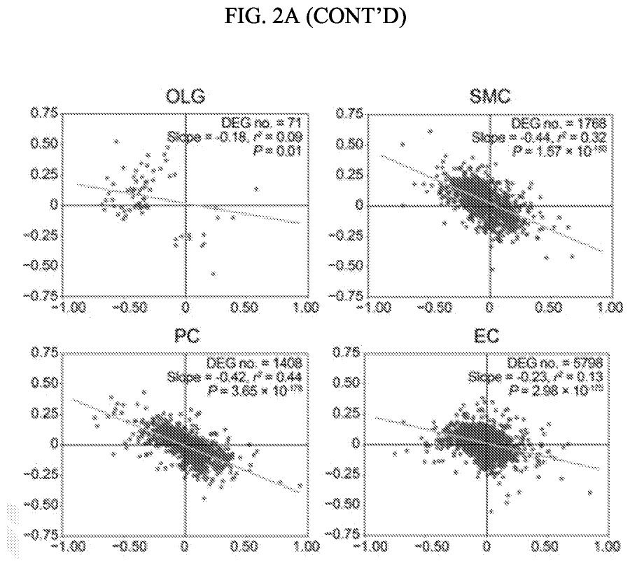

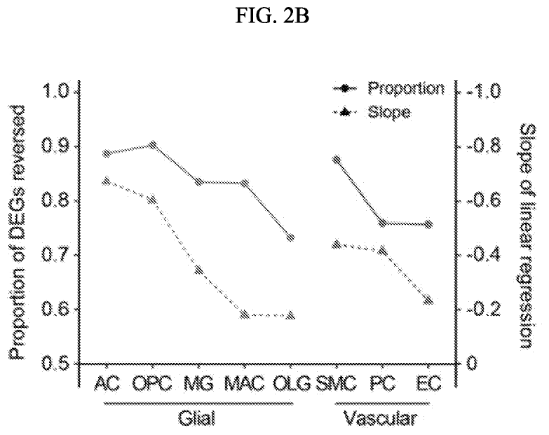

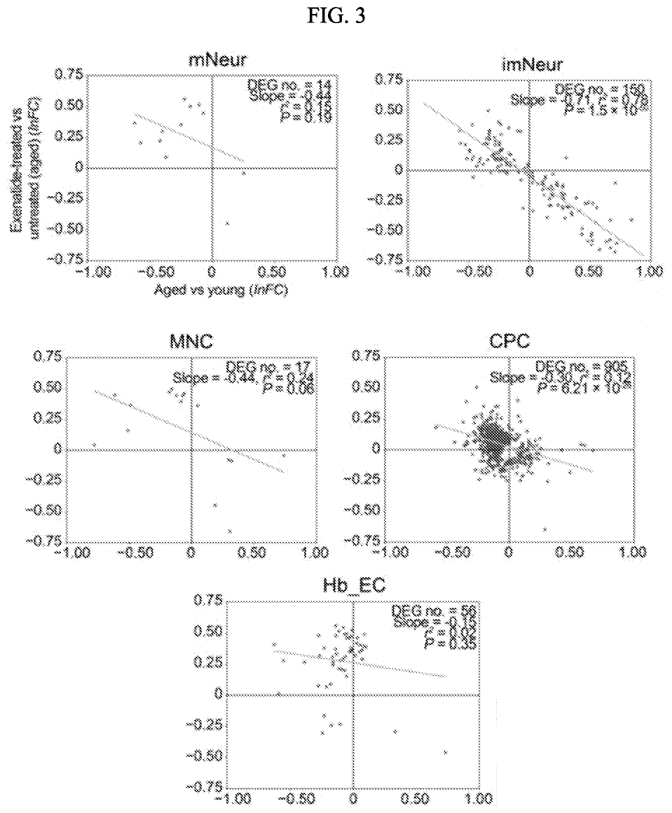

Single-cell transcriptomic profiling was performed in young adult, aged and exenatide-treated aged mice. The major cell type clusters identified and analyzed in mouse brains were visualized by UMAP as shown in FIGS. 1A-1D. Genome-wide expression changes in major cell types in aging brains and their modulation by GLP-1RA treatment was analyzed. Significant DEGs (defined as false discovery rate (FDR)-adjusted P-value <0.05) for each cell type were calculated to analyze their patterns of expression changes in aging and the effects of exenatide treatment. As shown in FIG. 2A, FIG. 2B, and FIG. 3, the aging-associated transcriptomic changes were universally reversed by exenatide treatment, across most of the major cell types in the brain.

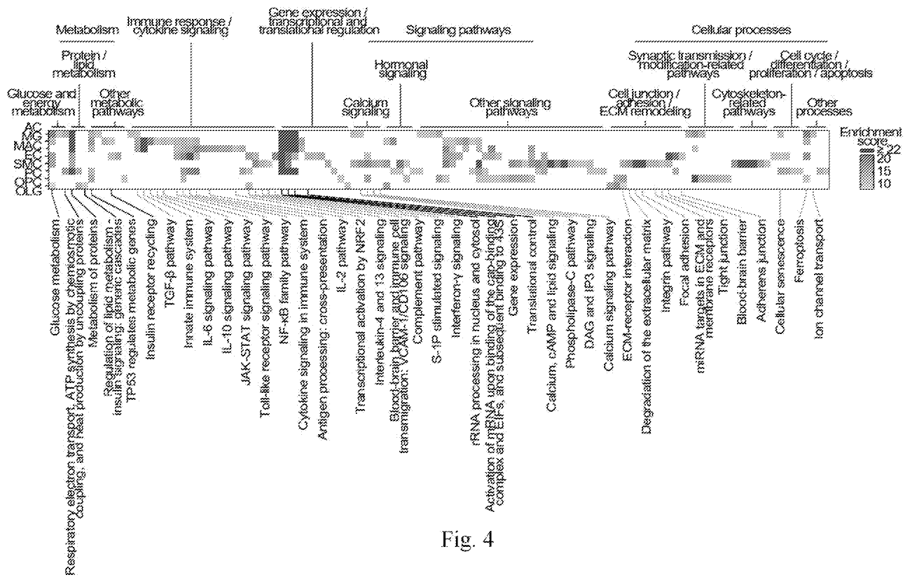

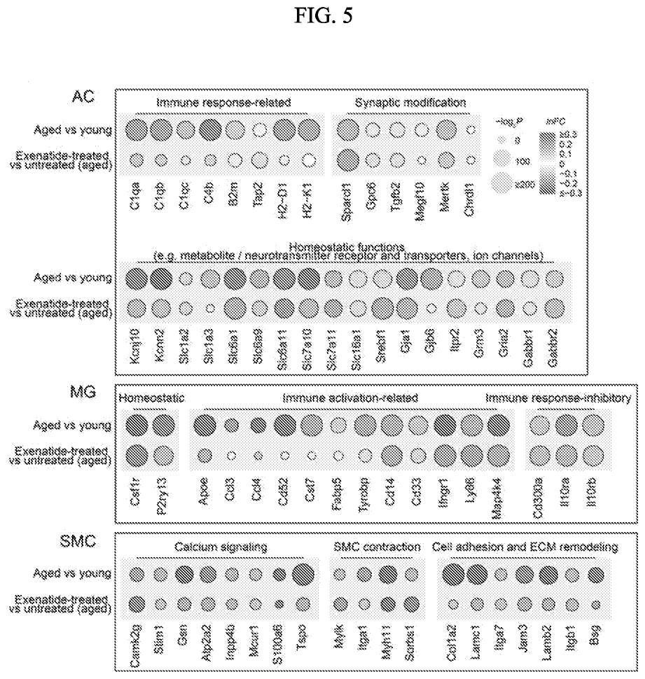

The expression changes reversed by GLP-1RA treatment are functionally relevant. Pathway enrichment analysis for each cell type on their most prominent reversed DEGs highlighted the amelioration of age-related expression changes involved in extensive cellular functions, as shown in FIG. 4. In most cell types, these included genes mediating glucose/energy, lipid and protein metabolic processes, as well as transcriptional and translational regulation. There are cell type-specific changes by pathway analysis. Furthermore, the expression changes of some genes are of significant functional roles. In ACs, age-related expression changes in genes mediating immune response, homeostatic functions and synaptic plasticity have been reported (Boisvert, M. M., G. A. Erikson, M. N. Shokhirev, N. J. Allen, The Aging Astrocyte Transcriptome from Multiple Regions of the Mouse Brain. Cell Reports 22, 269-285 (2018)). In this example, the ACs from GLP-1RA-treated mice appeared to partially revert to a younger phenotype, with downregulation of several complement 1 component genes (FIG. 5), and upregulation of subsets of genes encoding synaptic modification-related proteins, metabolite receptors and transporters, neurotransmitter receptors and ion channels (FIG. 5). On the other hand, immune response and cytokine signaling-related genes were especially prominent among DEGs reversed in MG and MAC, followed by EC (FIG. 5). Previous studies reported that microglia in the aged brain exhibit pro-inflammatory phenotypes (Hammond et al., Single-Cell RNA Sequencing of Microglia throughout the Mouse Lifespan and in the Injured Brain Reveals Complex Cell-State Changes. Immunity 50, 253-271.e6 (2019); A. F. Lloyd, V. E. Miron, The pro-remyelination properties of microglia in the central nervous system. Nature Reviews Neurology, 1-12 (2019)). After GLP-1RA treatment, the MGs showed an upregulation of multiple homeostatic function-related and immune response inhibitory genes, and reversed expression changes of several activation-associated genes (FIG. 5). In SMC, it was found post-treatment reversal of expression changes involving important functional processes associated with age-related vascular stiffening, such as calcium signaling, extracellular matrix (ECM) remodeling and contractile pathways (FIG. 4 and FIG. 5).

### Example 2—Exenatide Treatment Improves Cognitive Function in Aged Mice

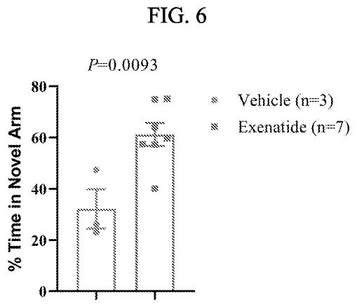

To assess the effect of exenatide to improve cognition in aged mice, 15-17 months old C57BL/6 were treated daily with saline vehicle (one male and two females; three mice in total) or 5 nmol/kg bw of exenatide (four males and three females; seven mice in total) for 2-4 months. Mice were subjected to Y maze test after treatment. As shown in FIG. 6, mice treated with exenatide spent significantly more time in the novel arm, which indicates an improvement of spatial memory.

### EXEMPLARY EMBODIMENTS

Embodiment 1. A method of treating a subject for aging-associated brain functional impairment, the method comprising administering to the subject an effective amount of a GLP-1R agonist whereby the aging-associated brain functional impairment is treated.

Embodiment 2. The method of embodiment 1, wherein the GLP-1R agonist is exenatide, liraglutide, lixisenatide, albiglutide, dulaglutide, semaglutide, taspoglutide, PF-06882961, OWL-833, TTP-273, or any other molecule that activates GLP-1R.

Embodiment 3. The method of embodiment 1, further comprising administering a pharmaceutically acceptable carrier with the GLP-1R agonist.

Embodiment 4. The method according to embodiment 1, wherein the subject is a mammal.

Embodiment 5. The method according to embodiment 4, wherein the mammal is a primate.

Embodiment 6. The method according to embodiment 5, wherein the primate is a human.

Embodiment 7. The method according to embodiment 1, wherein the subject is 50 years or older.

Embodiment 8. The method according to embodiment 1, wherein the aging-associated brain function impairment comprises cognitive impairment.

Embodiment 9. The method according to embodiment 8, wherein the cognitive impairment is any of attention and concentration, learning tasks and concepts, memory, information processing, visuospatial function, producing speech, understanding language, verbal fluency, problem solving, decision making, and executive functions.

Embodiment 10. The method according to embodiment 1, wherein the aging-associated brain function impairment comprises any of measurable structural, functional, and molecular changes of the brain.

Embodiment 11. The method according to embodiment 10, wherein the aging-associated measurable structural change is any of hippocampal atrophy, cortical atrophy, subcortical structural atrophy, cerebellar atrophy, global brain atrophy, microbleeds in the grey matter, microbleeds in the white matter, lacunes, infarcts, perivascular space, white matter intensity changes, and aggregation of protein species.

Embodiment 12. The method according to embodiment 11, wherein the protein species is any of amyloid beta, tau, alpha-synuclein, TAR DNA-binding protein 43, and prion.

Embodiment 13. The method according to embodiment 10, wherein the aging-associated measurable structural change is measured by an imaging method.

Embodiment 14. The method according to embodiment 13, wherein the imaging method is magnetic resonance imaging (MRI), computed tomography (CT), and/or ultrasonography (US).

Embodiment 15. The method according to embodiment 10, wherein the aging-associated measurable functional change is altered brain regional activation patterns, neuronal activity, functional connectivity, blood-brain barrier leakage, resting blood flow, glucose consumption, metabolite concentrations, neurovascular coupling, or functional hyperemia.

Embodiment 16. The method according to embodiment 15, wherein the aging-associated measurable functional change is measured by a functional imaging or recording method.

Embodiment 17. The method according to embodiment 16, wherein the functional imaging or recording method is functional magnetic resonance imaging (fMRI), magnetic resonance imaging (MRSI), hyperpolarized carbon-13 (¹³C) magnetic resonance spectroscopic imaging (MRSI), ultrasonography (US), positron emission tomography (PET), single-photon emission computerized tomography (SPECT), electroencephalography (EEG), magnetoencephalography (MEG), functional near-infrared spectroscopy (fNIRS), intracortical electrode recording, or deep brain electrode recording.

Embodiment 18. The method according to embodiment 10, wherein the aging-associated measurable molecular change is a change in gene expression; transcription of DNA; translation of RNA; location of DNA, RNA or protein in brain cells or tissue; or a secretion or release of proteins, DNA, RNA, mitochondria, cellular components, or mitochondrial components into blood fluids.

Embodiment 19. The method according to embodiment 10, wherein the aging-associated measurable molecular change is a change in cerebrospinal fluid (CSF) or blood/plasma compositions.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

Almanzar et al., A single-cell transcriptomic atlas characterizes ageing tissues in the mouse. Nature 583, 590-595 (2020).
Athauda et al., Exenatide once weekly versus placebo in Parkinson's disease: a randomised, double-blind, placebo-controlled trial. Lancet (London, England) 390, 1664-1675 (2017).
Boisvert, M. M., G. A. Erikson, M. N. Shokhirev, N. J. Allen, The Aging Astrocyte Transcriptome from Multiple Regions of the Mouse Brain. Cell Reports 22, 269-285 (2018).
Brauer et al., Diabetes medications and risk of Parkinson's disease: a cohort study of patients with diabetes. Brain (2020) https:/doi.org/10.1093/brain/awaa262.
Cai et al., Lixisenatide reduces amyloid plaques, neurofibrillary tangles and neuroinflammation in an APP/PS1/tau mouse model of Alzheimer's disease. Biochem Bioph Res Co 495, 1034-1040 (2018).
Cukierman-Yaffe et al., Effect of dulaglutide on cognitive impairment in type 2 diabetes: an exploratory analysis of the REWIND trial. Lancet Neurology 19, 582-590 (2020).
During et al., Glucagon-like peptide-1 receptor is involved in learning and neuroprotection. Nature Medicine 9, 1173-1179 (2003).
Gejl, et al., In Alzheimer's Disease, 6-Month Treatment with GLP-1 Analog Prevents Decline of Brain Glucose Metabolism: Randomized, Placebo-Controlled, Double-Blind Clinical Trial. Frontiers in aging neuroscience 8, 108 (2016).
Grieco et al., Glucagon-Like Peptide-1: A Focus on Neurodegenerative Diseases. Frontiers in Neuroscience vol. 13 (2019).
Hammond et al., Single-Cell RNA Sequencing of Microglia throughout the Mouse Lifespan and in the Injured Brain Reveals Complex Cell-State Changes. Immunity 50, 253-271.e6 (2019).
Holt et al., Preproglucagon Neurons in the Nucleus of the Solitary Tract Are the Main Source of Brain GLP-1, Mediate Stress-Induced Hypophagia, and Limit Unusually Large Intakes of Food. Diabetes 68, 21-33 (2018).
Isacson et al., The glucagon-like peptide 1 receptor agonist exendin-4 improves reference memory performance and decreases immobility in the forced swim test. European Journal of Pharmacology 650, 249-255 (2011))(1,2,3-5).
Lloyd, A. F. & V. E. Miron, The pro-remyelination properties of microglia in the central nervous system. Nature Reviews Neurology, 1-12 (2019)).
Lopez-Ofin et al. The hallmarks of aging. Cell vol. 153: 1194 (2013).
Mattson and Arumugan, Hallmarks of Brain Aging: Adaptive and Pathological Modification by Metabolic States. Cell Metabolism 27: 1176-1199 (2018).
Niccoli, T. & Partridge, L. Ageing as a risk factor for disease. Current Biology vol. 22 R741—R752 (2012).
Salcedo et al., Neuroprotective and neurotrophic actions of glucagon-like peptide-1: An emerging opportunity to treat neurodegenerative and cerebrovascular disorders. British Journal of Pharmacology. Vol. 166 1586-1599 (2012).
Vanlandewijck et al., Primary isolation of vascular cells from murine brain for single cell sequencing. Protocol Exchange https://doi.org/10.1038/protex.2017.159 (2018).
Yun et al., Block of A1 astrocyte conversion by microglia is neuroprotective in models of Parkinson's disease. Nature Medicine 24, 931-938 (2018).
Zhao et al., Pharmacologically reversible zonation-dependent endothelial cell transcriptomic changes with neurodegenerative disease associations in the aged brain. Nat Commun 11, 4413 (2020).

1. A method of treating a subject for aging-associated brain functional impairment, the method comprising administering to the subject an effective amount of a GLP-1R agonist whereby the aging-associated brain functional impairment is treated.

2. The method of claim 1, wherein the GLP-1R agonist is exenatide, liraglutide, lixisenatide, albiglutide, dulaglutide, semaglutide, taspoglutide, PF-06882961, OWL-833, or TTP-273, or any other molecule that activates GLP-1R.

3. The method of claim 1, further comprising administering a pharmaceutically acceptable carrier with the GLP-1R agonist.

4. The method according to claim 1, wherein the subject is a mammal.

5. The method according to claim 4, wherein the mammal is a primate.

6. The method according to claim 5, wherein the primate is a human.

7. The method according to claim 1, wherein the subject is 50 years of age or older.

8. The method according to claim 1, wherein the aging-associated brain function impairment comprises cognitive impairment.

9. The method according to claim 8, wherein the cognitive impairment is any of attention and concentration, learning tasks and concepts, memory, information processing, visuospatial function, producing speech, understanding language, verbal fluency, problem solving, decision making, and executive functions.

10. The method according to claim 1, wherein the aging-associated brain function impairment comprises any of measurable structural, functional, and molecular changes of the brain.

11. The method according to claim 10, wherein the aging-associated measurable structural change is any of hippocampal atrophy, cortical atrophy, subcortical structural atrophy, cerebellar atrophy, global brain atrophy, microbleeds in the grey matter, microbleeds in the white matter, lacunes, infarcts, perivascular space, white matter intensity changes, and aggregation of protein species.

12. The method according to claim 11, wherein the protein species is any of amyloid beta, tau, alpha-synuclein, TAR DNA-binding protein 43, and prion.

13. The method according to claim 10, wherein the aging-associated measurable structural change is measured by an imaging method.

14. The method according to claim 13, wherein the imaging method is magnetic resonance imaging (MRI), computed tomography (CT), and/or ultrasonography (US).

15. The method according to claim 10, wherein the aging-associated measurable functional change is altered brain regional activation patterns, neuronal activity, functional connectivity, blood-brain barrier leakage, resting blood flow, glucose consumption, metabolite concentrations, neurovascular coupling, or functional hyperemia.

16. The method according to claim 15, wherein the aging-associated measurable functional change is measured by a functional imaging or recording method.

17. The method according to claim 16, wherein the functional imaging or recording method is functional magnetic resonance imaging (fMRI), magnetic resonance imaging (MRSI), hyperpolarized carbon-13 (¹³C) magnetic resonance spectroscopic imaging (MRSI), ultrasonography (US), positron emission tomography (PET), single-photon emission computerized tomography (SPECT), electroencephalography (EEG), magnetoencephalography (MEG), functional near-infrared spectroscopy (fNIRS), intracortical electrode recording, or deep brain electrode recording.

18. The method according to claim 10, wherein the aging-associated measurable molecular change is a change in gene expression; transcription of DNA; translation of RNA; location of DNA, RNA or protein in brain cells or tissue; or a secretion or release of proteins, DNA, RNA, mitochondria, cellular components, or mitochondrial components into blood fluids.

19. The method according to claim 10, wherein the aging-associated measurable molecular change is a change in cerebrospinal fluid (CSF) or blood/plasma compositions.
